(19) United States
(12) Patent Application Publication
(10) Pub. No.: US 2011/0104795 A1
(43) Pub. Date: May 5, 2011

(54) Compost Turning and Aerating Machine

(75) Inventors: Merle L. Ranck, Gap, PA (US); Duane E. Newswanger, Conestoga, PA (US)

(73) Assignee: Terra-Gro, Inc.

(21) Appl. No.: 12/610,086

(22) Filed: Oct. 30, 2009

(51) Int. Cl. C12M 1/02

(52) U.S. Cl. 435/290.2

(57) ABSTRACT

A compost windrow turning machine disintegrates an existing windrow of compost material, conveys the disintegrated material rearwardly, aerates the rearwardly conveyed material with a positive displacement of air into the rearwardly conveyed compost material, and re-forms a new windrow of remixed and aerated compost material rearwardly of the machine in line with the original windrow. The disintegration of the original compost windrow is accomplished with a rotor having segmented teeth arranged in a spiral to shred the original compost material and convey the disintegrated material to a central chain and slat conveyor positioned behind the rotor. A positive displacement of air is injected into the compost material on the central conveyor by a pair of laterally disposed fans mounting in conduits angled inwardly toward the central conveyor. The discharge of material off the central conveyor is passed through a set of windrow-forming shields to create a new windrow.

### FIELD OF THE INVENTION

The present invention relates generally to a machine for turning windrows of compost material, and, more particularly, to an apparatus that injects air into the compost windrow as the compost is being conveyed through the machine.

### BACKGROUND OF THE INVENTION

Composting is well recognized as an environmentally sound and economical means of recycling a variety of organic, biodegradable materials. Various composting technologies are available, including static pile composting, windrow composting, aerated-windrow composting, and in-vessel composting employing, for example, circular reactors, horizontal agitated bay or bin reactors and vertical reactors.

Biodegradable materials can be composted on a mass production basis by arranging the biodegradable material being composted into long stacks, or windrows that are placed within shelters to keep moisture off of the windrows. Generally, the windrows of compost can have a width of ten to fifteen feet, a height of six to eight feet and a length of hundreds of feet with the top surface being generally rounded. Periodically, at least twice each week, it is necessary to turn, or more accurately re-mix, the windrow of biodegradable material being turned into compost. This re-mixing of the windrow serves to incorporate air into the windrow to facilitate the aerobic process that is taking place to convert the biodegradable materials into compost. In addition, the re-mixing of the compost materials serves to redistribute the microbiological agents, and break up the partially decomposed materials into smaller particles to further facilitate the aerobic process that biologically degrades the material into compost. Improved aeration of the windrow of compost material will hasten the aerobic process.

Known compost turning machines engage the long windrow of compost material and elevate the material before reforming the windrow usually within the line of the windrow being remixed. Examples of such machines can be found in U.S. Pat. No. 5,387,036, issued to Daniel Hagen, et al on Feb. 7, 1995; in U.S. Pat. No. 5,395,417, issued to Thomas Thomas, et al on Mar. 7, 1995; and in U.S. Pat. No. 5,459,071, issued to Larry Finn on Oct. 17, 1995. Each of these machines are very large and expensive devices that engage the elongated windrow of compost material with a rotor or an auger that breaks up the windrowed compost material and conveys the compost material into another pile. In each of these large windrow turning machines, the remixed windrow of compost is re-deposited in line in the same line as the windrow before being engaged by the machine, although spaced by the operating width of the machine.

In U.S. Pat. No. 4,478,520, granted on Oct. 23, 1984, to Herbert Cobey, the compost turning machine is formed with a pair of stub augers mounted to the opposing sides of a central and rearwardly disposed rotor formed with segmented teeth to break up the compost material in the windrow and convey the material rearwardly. A different self-propelled machine configuration is found in U.S. Pat. No. 5,191,754, granted on Mar. 9, 1993, to Norval Morey. In the Morey apparatus, a header formed of a pair of fixed augers that convey the compost material into a blower that blows the disintegrated compost material rearwardly through a spout positioned over the top of the machine. While the Morey apparatus can achieve significant re-mixing and aeration due to being blown rearwardly through the spout, the Morey apparatus would be prone to plugging and would not have a positive control over the material being discharged from the spout.

Pull-type compost turning machines have also been developed, as is represented in U.S. Pat. No. 3,982,772, granted to Werner Scherer on Sep. 28, 1976, in which an auger header engages a small windrow of compost material and conveys the material rearwardly with sufficient velocity to engage a pair of windrow forming shields to re-form the windrow behind the machine. The pull-type machine in U.S. Pat. No. 4,932,196, issued on Jun. 12, 1990, to Bradley Schnittjer, is formed with three chain and slat conveyors that engage the windrow of compost to elevate the material and convey the material rearwardly. A pair of lateral conveyors serves to consolidate the material being conveyed by the outside chain and slat conveyors to form a new windrow behind the central conveyor.

All of these known prior art machines attempt to aerate the compost material as part of the re-mixing process by disintegrating the existing pile of compost material and elevating the material to be dropped, or blown, into a new pile rearwardly of the machine. None of these machines provide a positive air displacement into the compost material between being disintegrated at the original pile and being re-deposited into the new pile. One known pull-type machine found in U.S. Pat. No. 5,309,703, granted to William Brinton on May 10, 1994, does attempt to insert a flow of air into the compost material as the material is being conveyed to the newly formed pile. The Brinton apparatus incorporates a series of conduits over the conveying mechanism to discharge air or oxygen through holes formed in the conduits into the compost material in an attempt to aerate the compost material before being re-deposited into a new pile.

It would be desirable to provide a machine that would be effective in the re-mixing of the compost material from an original pile into a newly formed pile to maximize the effective aeration of the compost material, but also to provide a machine that would provide a positive displacement of air into the material being conveyed rearwardly to form the new windrow of remixed compost material.

### SUMMARY OF THE INVENTION

It is an object of this invention to provide a machine to remix and aerate a windrow of compost material.

It is a feature of this machine that a positive displacement of air is moved into the flow of compost material being conveyed rearwardly for re-forming into a new windrow of compost material.

It is an advantage of this invention that the positive displacement of air provides a significant forced aeration of the compost material.

It is another advantage of this invention that the positive displacement of air into the remixing of the compost material speeds the composting process.

It is another feature of this invention that the shredding header that engages the original windrow of compost material aggressively shreds the compost material into a substantially uniform flow of disintegrated compost material to be re-formed into a new windrow.

It is still another feature of this invention that the machine for turning compost windrows is self-propelled with an enclosed, environmentally sealed operator's cab.

It yet another feature of this invention that a positive displacement of air is injected into the flow of disintegrated compost material being conveyed rearwardly by a pair of fans drawing ambient air into conduits directing the forced air flow into the rearward conveyor.

It is still another advantage of this invention that the windrow engaging header is formed with segmented teeth to effectively shred the original windrow of compost material to be conveyed rearwardly by a central conveyor.

It is another feature of this invention that the individually mounted teeth are oriented at an angle to the axis of rotation of the rotor so that the compost is displaced toward the centerline of the machine when conveyed rearwardly by the operation of the rotor.

It is still another feature of this invention that the individual teeth are mounted on the rotor in a spiral configuration that is mirrored about the center of the rotor.

It is another advantage of this invention that the inward direction of the compost material conveyed by the operation of the rotor helps to affect a mixing of the compost material before being re-deposited into a new windrow behind the machine by the central conveyor.

It is still another feature of this invention that the header is mounted on the forward end of the central conveyor.

It is yet another advantage of this invention that the vertical movement of the header to engage the windrow of compost material will move the forward end of the central conveyor vertically in conjunction with the vertical movement of the header.

It is a further advantage of this invention that the spatial relationship between the rotor and the forward end of the central convey remains constant irrespective of the vertical position of the header.

It is another feature of this invention that the machine engages the compost material in front of the machine and deposits the mixed and aerated windrow immediately behind the machine.

It is still a further advantage of this invention that spacing between windrows of compost material in an enclosed shelter can be minimized as the machine does not require space between the windrows of compost material to operate.

It is a further feature of this invention that the rearward end of the central conveyor discharges the disintegrated compost material through winnowing shields to direct the flow of compost material being discharged off the central conveyor into a re-formed windrow.

It is another object of this invention to provide a compost windrow turning and aeration machine that is durable in construction, inexpensive of manufacture, carefree of maintenance, facile in assemblage, and simple and effective in use.

These and other objects, features and advantages are accomplished according to the instant invention by providing a compost windrow turning machine that moves into an existing windrow of compost material to disintegrate the original pile of compost material, convey the disintegrated material rearwardly, aerate the rearwardly conveyed material with a positive displacement of air into the flow of compost material, and re-form a new windrow of remixed and aerated compost material rearwardly of the machine in line with the original windrow. The disintegration of the original compost windrow is accomplished with a rotor having segmented teeth arranged in a spiral to shred the original compost material and convey the disintegrated material to a central chain and slat conveyor positioned behind the rotor. A positive displacement of air is injected into the compost material on the central conveyor by a pair of fans mounting in conduits angled inwardly toward the central conveyor. The discharge of material off the central conveyor is passed through a set of windrow-forming shields to create a new windrow.

### BRIEF DESCRIPTION OF THE DRAWINGS

The foregoing and other objects, features, and advantages of the invention will appear more fully hereinafter from a consideration of the detailed description that follows. It is to be expressly understood, however, that the drawings are for illustrative purposes and are not to be construed as defining the limits of the invention.

FIG. 1 is a right, front perspective view of the compost turning and aerating machine incorporating the principles of the instant invention, with the header in a lowered position for engaging an elongated pile of compost material;

FIG. 2 is a left, front perspective view of the compost turning machine depicted in FIG. 1;

FIG. 3 is a right side elevational view of the compost turning machine;

FIG. 4 is a front elevational view of the compost turning machine;

FIG. 5 is a left side elevational view of the compost turning machine;

FIG. 6 is a rear elevational view of the compost turning machine;

FIG. 7 is a top plan view of the compost turning machine;

FIG. 8 is a cross-sectional view through the right side of the compost turning machine corresponding to lines 8-8 of FIG. 6 to show the support link for the rear of the central conveyor and the mounting of the header on the front end of the central conveyor;

FIG. 9 is a cross-sectional view through the right side of the compost turning machine corresponding to lines 9-9 of FIG. 7 to show the relationship between the header and the central conveyor;

FIG. 10 is a perspective cross-sectional view through the right side of the compost turning machine corresponding to lines 10-10 in FIG. 1; and

FIG. 11 is a cross-sectional view taken along a horizontal plane corresponding to lines 11-11 in FIG. 3 to show the relationship of the air inlet ducts to the central conveyor.

### DETAILED DESCRIPTION OF THE PREFERRED EMBODIMENT

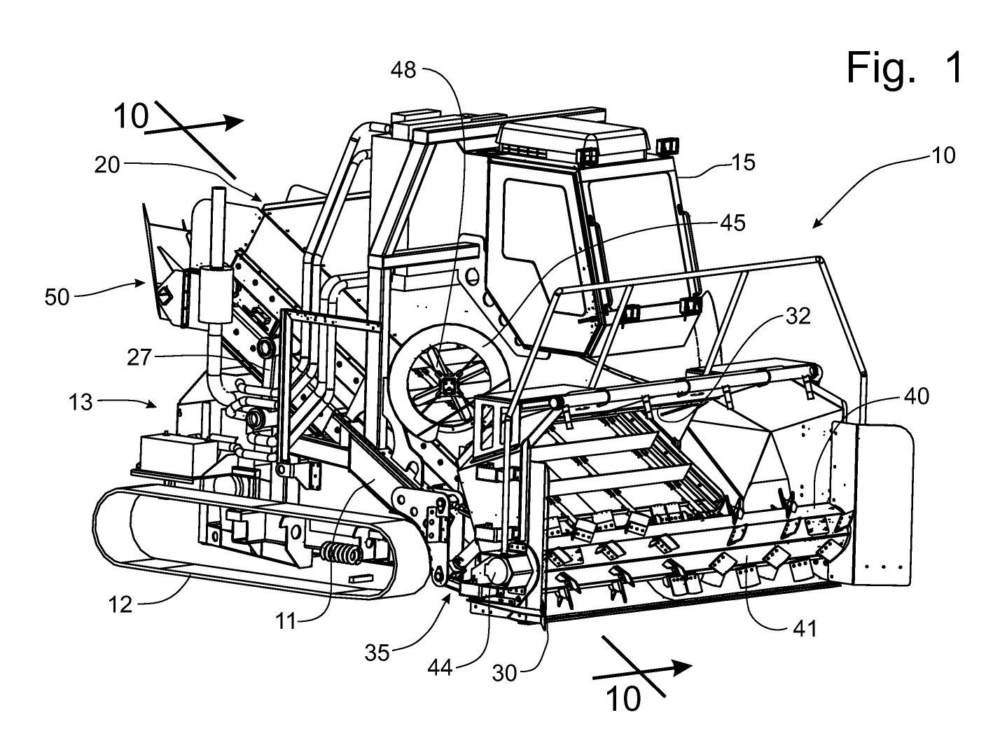

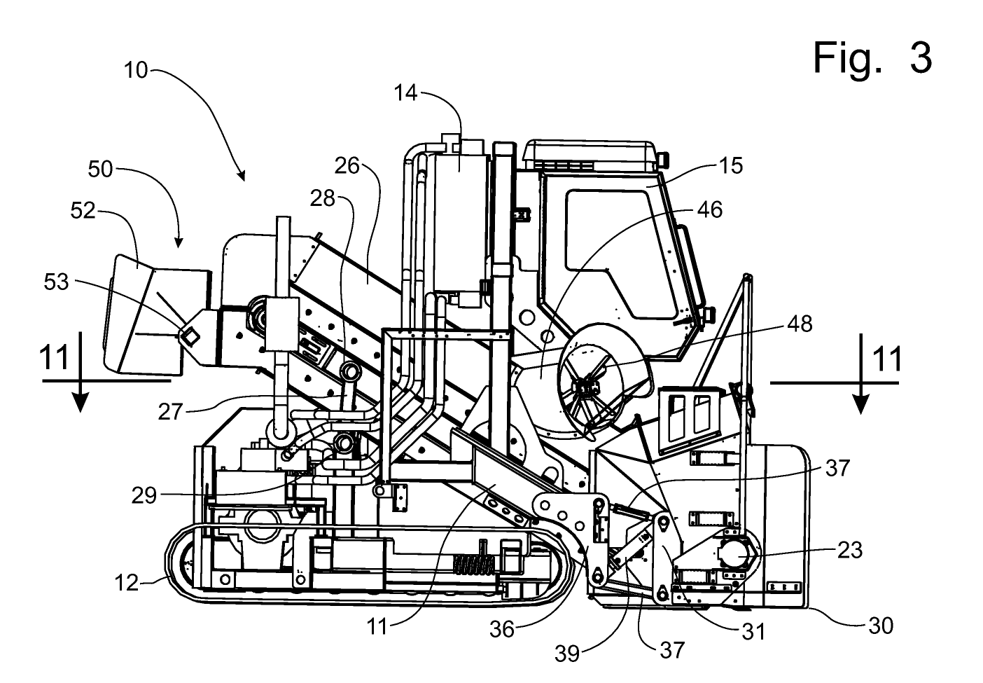

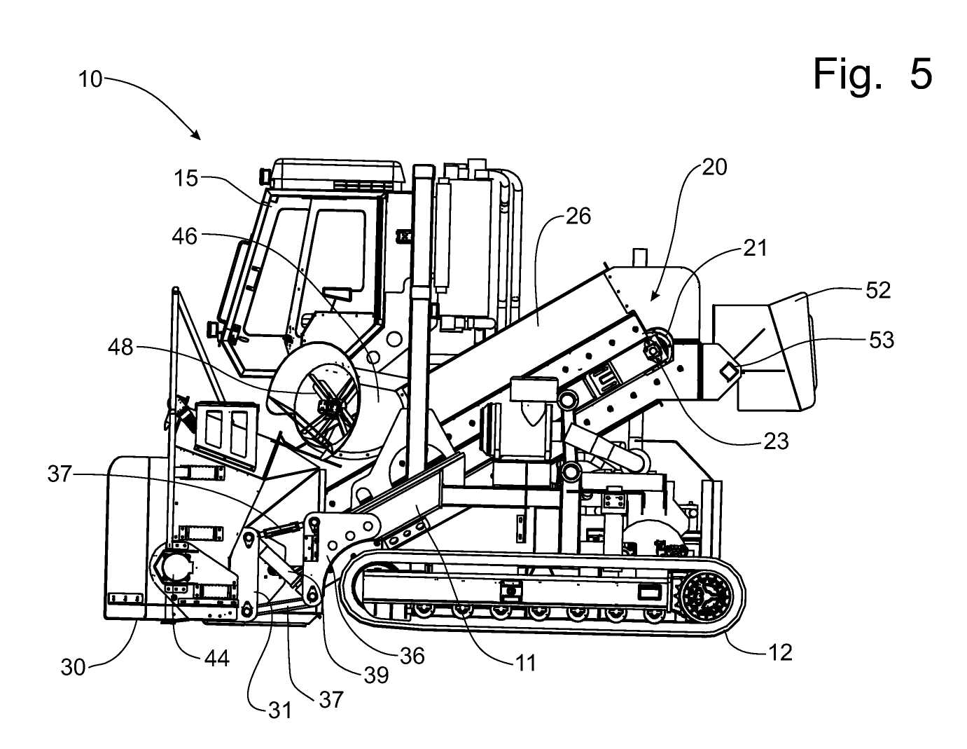

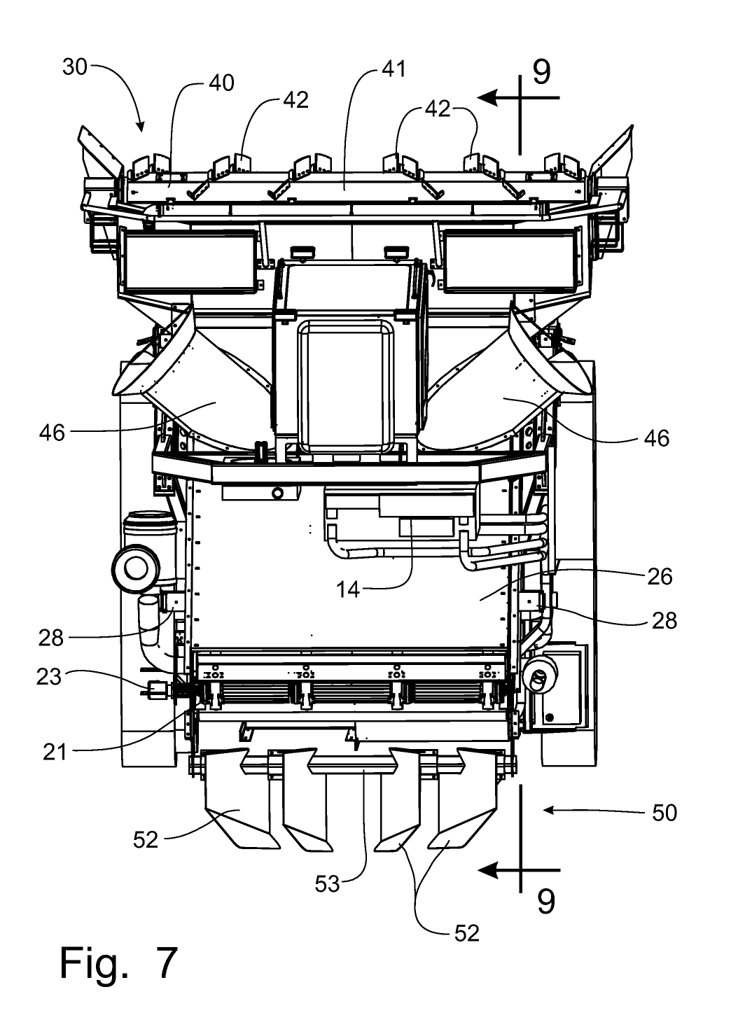

Referring to FIGS. 1-7, a compost turning and aerating machine incorporating the principles of the instant invention can best be seen. Any left, right, front and rear references are used as a matter of convenience and are determined by standing at the rear of the machine where the elevated central conveyor discharges material from the machine and facing forwardly toward the header at the front of the machine, which is the normal forward direction of travel of the machine during operation thereof. The machine 10 is preferably a self-propelled machine having a main frame 11 supported on tracks 12 for the mobile support of the machine 10 over the surface of the ground. One skilled in the art will recognize that while the tracks 12 are preferable for support and movement of the machine 10, other devices, such as conventional wheels and tires, can also provide mobile support of the machine.

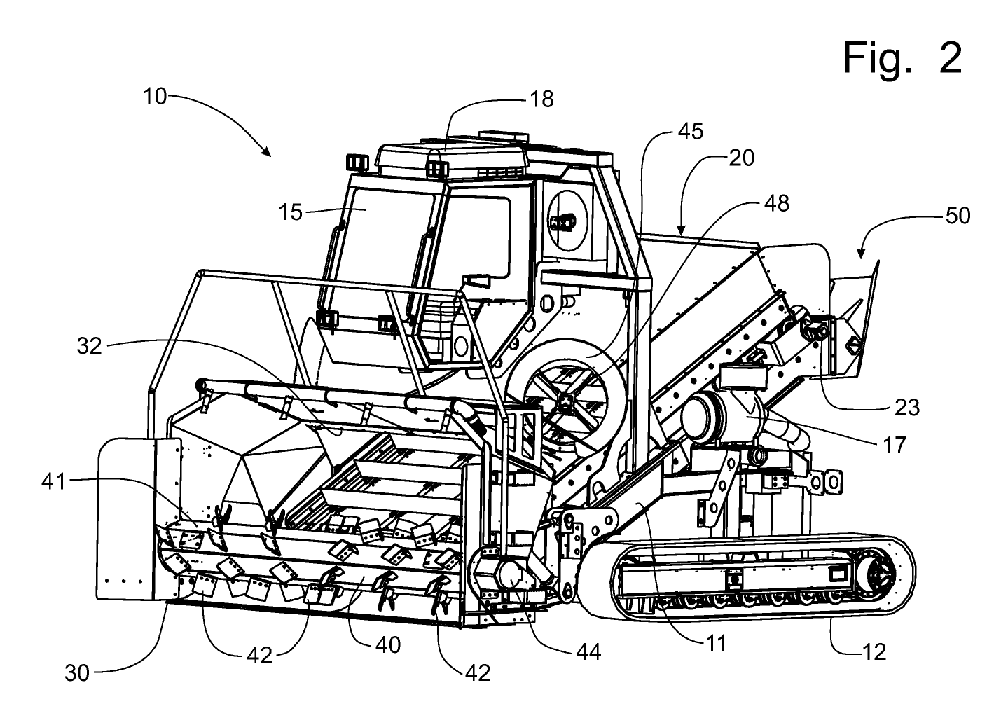

An engine 13 is mounted on the frame, preferably beneath the rear of an elevated central conveyor 20 described in greater detail below. The engine 13 is connected to a coolant radiator 14 located in an elevated position above the central conveyor 20 to minimize the effect of dust from the operation of the machine. Charge air for the operation of the engine is provided through the main air filter 17, best seen in FIG. 2, to provide a source of combustion air to the engine through an engine turbocharger (not shown). Air into the operator's cab 15 located forwardly on the frame 11 above the header 30 and the front end of the central conveyor 20 is provided through a roof mounted air conditioner 18 and associated filtration apparatus (not shown). The machine 10 is provided with conventional power transmission apparatus operatively interconnecting the engine 13 and the tracks 12 to transmit operative power thereto, and to the operable components of the machine 10, including the central conveyor 20, the header 30, and the air input apparatus 45. Preferably, this power transmission apparatus will include a hydraulic system 16 that provides a source of hydraulic fluid under pressure to the hydraulic power components described in greater detail below.

As a matter of general operation of the machine 10, the header 30 is positioned to engage an elongated pile of compost material in need of remixing and aerating. The header 30 disintegrates the pile of compost material by breaking any clumped material into component pieces and conveys the disintegrated compost material rearwardly into a central conveyor 20 that elevates the collected material for discharge from the rear of the conveyor 20, which is elevated above the header 30, to redeposit the compost material into a remixed pile behind the machine 10. While the compost material is being elevated on the central conveyor 20, the air input apparatus 45 directs a flow of air onto the central conveyor 20 so that the air will mix with the compost material being conveyed and discharged off the rearward end of the conveyor 20 to aerate the compost material as the compost material is being remixed.

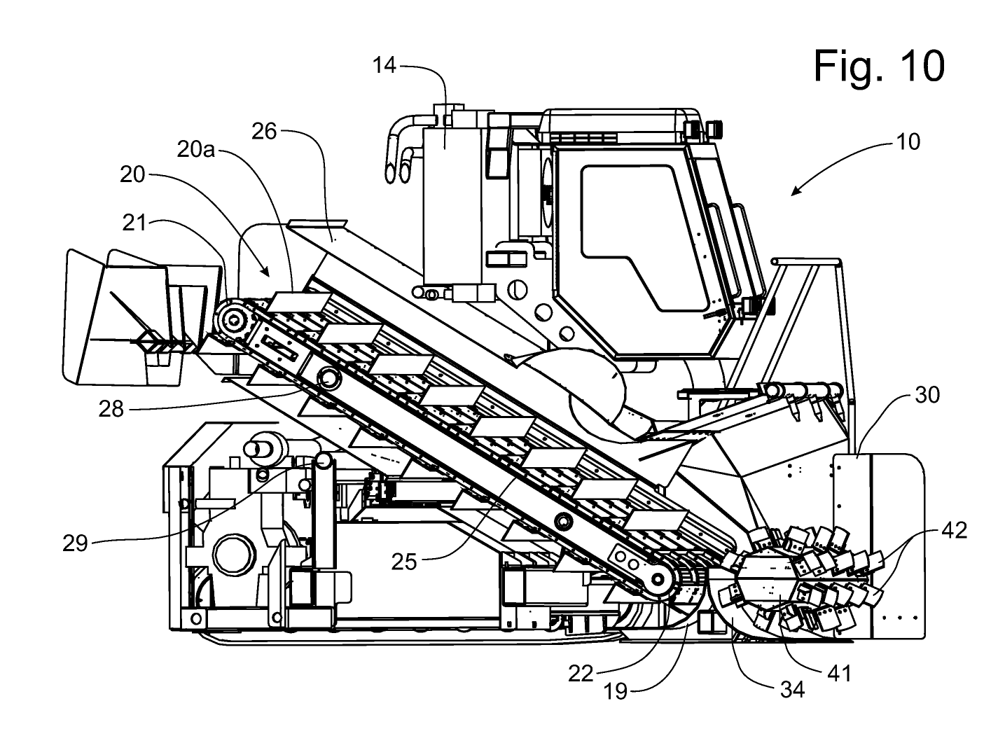

The central conveyor is best seen in FIGS. 1-5 and 8-11 and is formed of a chain and slat conveyor mechanism positioned between the tracks 12. The central conveyor 20 is oriented such that the conveyor elevates the compost material placed thereon from the front of the conveyor to the discharge end at the rear of the machine 10. Preferably, the compost material is discharged off the rear of the conveyor 20 into a windrow-forming device 50 having a plurality of shields 52 fixed on a transverse support shaft 53. The shields 52 are oriented so as to be angled toward the longitudinal centerline of the machine 10, which will serve to deflect the discharged compost material into a centralized pile rearwardly of the machine 10.

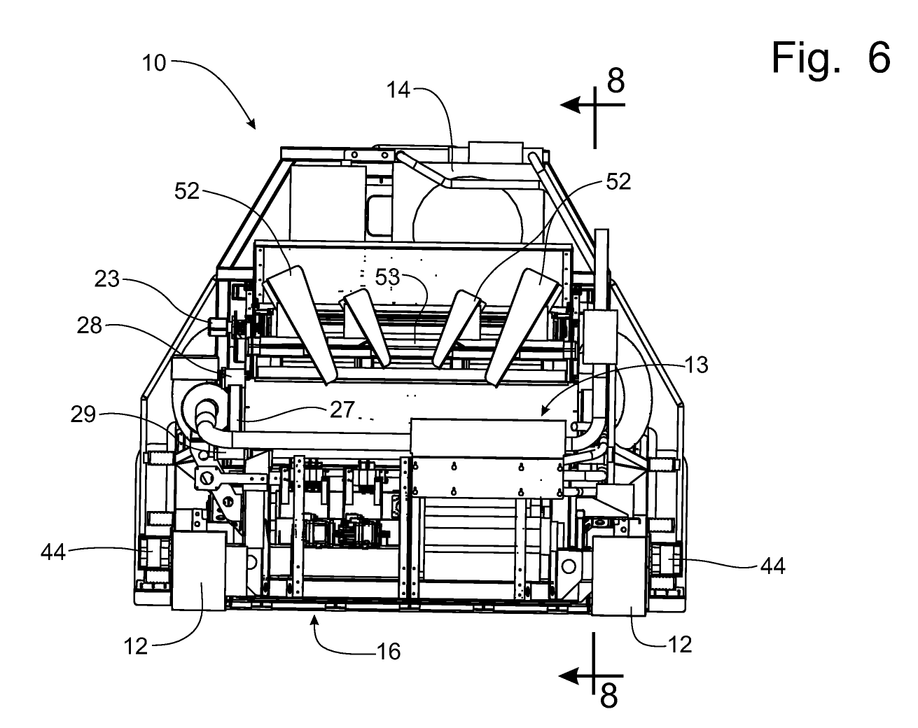

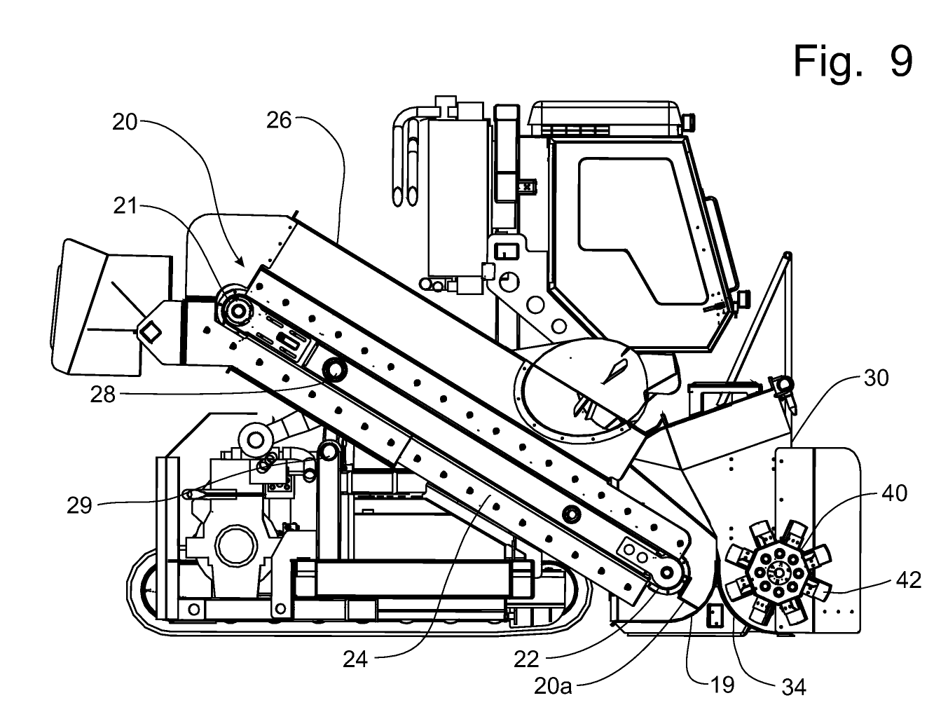

The chain and slat conveyor 20 is entrained around a drive sprocket assembly 21 at the rear of the conveyor 20 and a driven sprocket assembly 22 at the front of the conveyor 20. The drive sprocket assembly 21 is powered by a hydraulic motor 23, shown in FIGS. 2 and 6 as being on the left side of the machine 10, operatively connected to the hydraulic system 16. As seen best in FIG. 9, the sprocket assemblies 21 and 22 are mounted on side frame members 24, the drive sprocket assembly 21 being positionally adjustable to maintain proper tension in the chain and slat conveyor 20. The side frame members 24 support a central floor member 25 over which the rearwardly moving run of the chain and slat conveyor 20 moves to convey the compost material rearwardly. The front of the conveyor 20 cooperates with a curved member 19 so that the slats 20a will scoop material conveyed rearwardly by the header 30 and move the compost material rearwardly over the floor 25.

The rearward portion of the central conveyor 20 is supported from a pair of laterally spaced vertical support links 27 pivotally connected at the upper end by a pivot 28 to the respective side frame members 24 and at the lower end by a transverse pivot shaft 29 that passes beneath the conveyor 20. The front portion of the central conveyor 20 mounts the header 30, as will be described in greater detail below, and is vertically movable with the header 30 to allow the header 30 to engage the pile of compost material. As the header 30 and the front end of the conveyor 20 move vertically, the side frame members 24 pivot about the pivot connection 28. However, the lift mechanism 35 for the header 30 operates to move the header 30 in a substantially straight vertical line. As a result, the rearward end of the conveyor 20 must move longitudinally to accommodate this induced movement of the header 30. The pivotal movement of the support links 27 about the transverse pivot shaft 29 allows the rearward end of the conveyor 20 to shift slightly rearwardly as the front end of the conveyor 20 moves vertically.

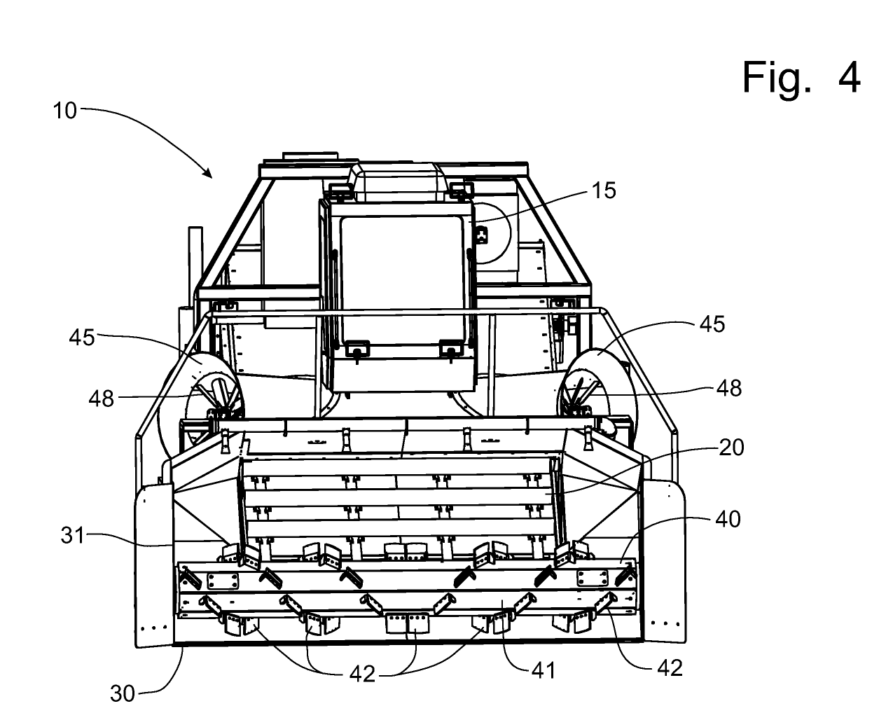

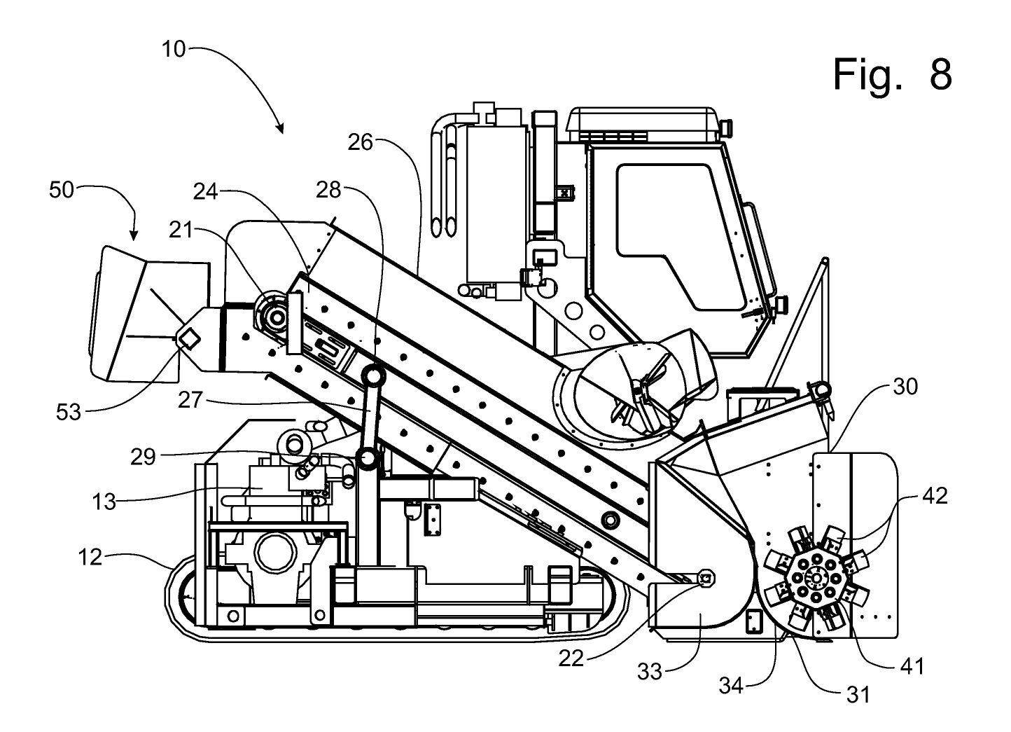

The header 30 is formed with a header frame 31 that rotatably supports a transverse rotor 40. The frame 31 defines a box-like structure with a central discharge opening 32 in register with the central conveyor 20. As is best seen FIG. 4, the back wall of the header frame is sloped to direct the flow of disintegrated compost material toward the central discharge opening 32. As is shown in FIG. 8, the header frame 31 is mounted to the front end of the central conveyor 20 with the axis of rotation of the driven sprocket assembly 22 being secured within a mounting flange 33 so that the header frame 31 and the front end of the conveyor 20 will move together. Referring now to FIGS. 1-3 and 5, the header 30 is secured to the frame 11 of machine 10 by a lift mechanism 35 on each opposing side of the header 30. A mounting bracket 36 is affixed to the frame 11 to support each lift mechanism 35 which includes a four-bar linkage 37 on opposing sides of the header 30, interconnecting the header frame 31 and the mounting bracket 36 that induces a vertical movement of the header frame 31 along a substantially straight vertical line. A hydraulic cylinder 39 is anchored on each mounting bracket 36 and is connected to the header frame 31 so that the extension of the hydraulic cylinders 39 pushes the header 30 vertically, taking the front end of the conveyor 20 with the header 30.

In operation, the operator moves the header 30 vertically, as needed, to engage the pile of compost material. Since the axis of the driven sprocket assembly 22 is supported on the mounting flange 33 of the header, the vertical movement of the header 30 caused by the expansion and contraction of the hydraulic cylinders 39 also moves the front end of the conveyor 20 with the header 30. The mounting of the side frame members 24 on the support links 27, as described above, allows the conveyor 20 to pivot vertically about the pivot connections 28 and to swing longitudinally by virtue of the pivotal mounting of the support links 27 on the transverse pivot shaft 29.

The rotor 40 can be a cylindrical tubular member, but is preferably fabricated into an octagonally-shaped cylinder 41 to facilitate the mounting of segmented teeth 42 onto the flat surfaces of the cylinder 41. The opposing ends of the rotor 40 are drivingly connected to hydraulic motors 44 operably associated with the hydraulic system 16 to power the rotation of the rotor 40 in a manner to drive the segmented teeth 42 into the pile of compost material to be disintegrated. The teeth 42 are mounted on the rotor cylinder 41 at an angle to the axis of rotation of the rotor 40 and the teeth 42 are arranged in a spiral configuration around the cylinder 41, similar to that of a segmented auger flighting. With this configuration, the rotation of the rotor 40 will cut the segmented teeth 42 into the pile of compost material to disintegrate the compost material from the pile and convey the disintegrated material rearwardly and inwardly toward the center of the rotor. Thus, the rotor 40 operates to convey the disintegrated compost material through the central discharge opening 32 into engagement with the central conveyor 20.

Referring now to FIGS. 8 and 9, the header frame 31 is formed with a curved member 34 cooperable with the segmented teeth 42 to permit the teeth 42 to elevate the disintegrated compost material for discharge from the header 30 through the central opening 32 into the conveyor 20.

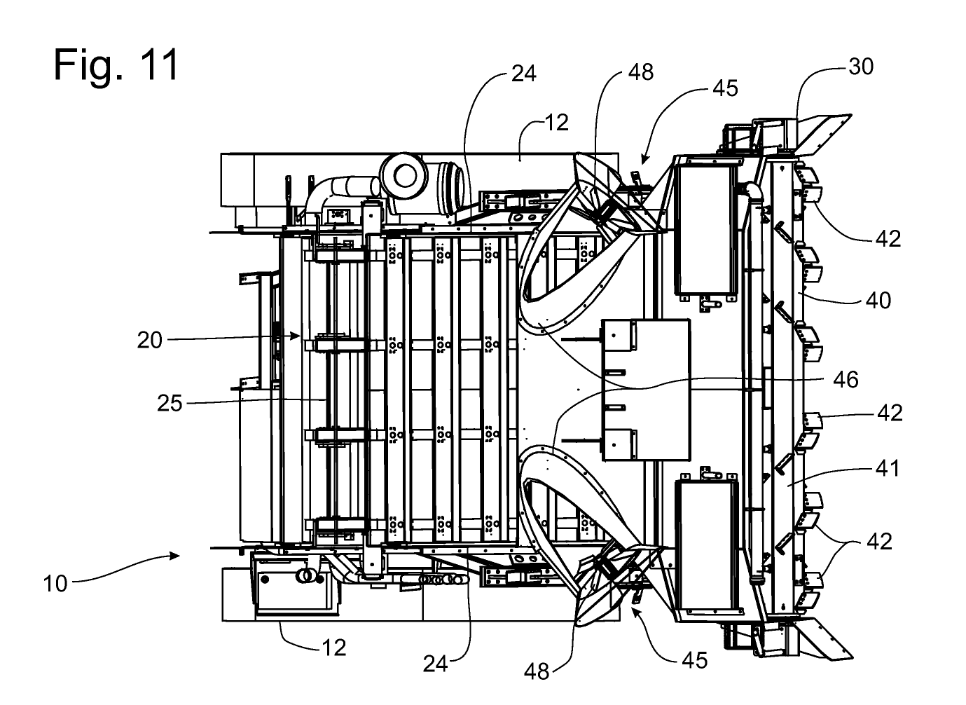

To enhance the aeration of the compost material as the material is being remixed and re-deposited onto a new pile rearwardly of the machine 10, the machine 10 is provided with a pair of laterally spaced air input devices 45. The air input devices 45 include a generally cylindrical duct 46 that is oriented inwardly from an inlet opening located above the front portion of the central conveyor 20. A fan 48 is mounted at the inlet opening to draw air from outside of the machine 10 into the duct 46 which directs the flow of air onto the top of the central conveyor 20 between the respective lateral edges of the conveyor 20 and the longitudinal centerline thereof, as is best seen in the cross-sectional view of FIG. 11. The flow of air induced by the rotating fans 48 will carry rearwardly with the compost material on the conveyor 20, contained by the shielding 26 around the conveyor 20 to the rear discharge end of the conveyor 20 to be mixed with the compost material being re-deposited into a windrow by the windrow-forming apparatus 50.

The invention of this application has been described above both generically and with regard to specific embodiments. Although the invention has been set forth in what is believed to be the preferred embodiments, a wide variety of alternatives known to those of skill in the art can be selected within the generic disclosure. The invention is not otherwise limited, except for the recitation of the claims set forth below.

Having thus described the invention, what is claimed is:

1. A machine for turning and aerating an elongated pile of compost material, comprising:
a mobile frame adapted for movement over the ground;
a central conveyor supported on said frame and oriented to convey material rearwardly and upwardly for discharge therefrom rearwardly of said conveyor;
a header mounted forwardly on said frame to engage and collect said compost material, said header delivering collected compost material to said central conveyor; and
air input apparatus supported on said frame to direct a flow of air onto the conveyor as compost material is being conveyed rearwardly thereby.

2. The machine of claim 1 wherein said air input apparatus includes a pair of ducts angled inwardly from an inlet opening toward a longitudinal centerline of said conveyor.

3. The machine of claim 2 wherein each said duct has a fan mounted at the corresponding said inlet opening to draw air from outside said machine into said conveyor.

4. The machine of claim 3 wherein said conveyor is formed with shielding enclosing an upper portion of said conveyor such that the flow of air created by said air input apparatus is mixed with said compost material being conveyed by said conveyor when discharged from said conveyor rearwardly of said machine.

5. The machine of claim 3 wherein said header includes a transversely extending rotor having teeth mounted thereon to remove compost material from said pile, said rotor being operable to convey said compost material to said conveyor.

6. The machine of claim 5 wherein said conveyor has a forward end and a rearward end with said forward end being mounted on said header, said header including a lift mechanism operable to move said header vertically, said front end of said conveyor moving vertically with said header.

7. The machine of claim 6 wherein said conveyor is connected to a pair of laterally spaced support links, each said support link being pivotally connected to said conveyor and pivotally supported on said frame.

8. The machine of claim 7 wherein said lift mechanism includes a four-bar linkage that induces a generally linear vertical movement of said header, said support links being operable to allow said rearward end moving longitudinally through the pivotal support provided by said support links.

9. The machine of claim 8 wherein each of said teeth is individually mounted on a rotor cylinder in an angled orientation with respect to a longitudinal centerline of said machine, said teeth being mounted on said rotor cylinder in a spiral configuration to facilitate movement of compost material through a central discharge opening in said header to said conveyor.

10. The machine of claim 6 further comprising a windrow-forming apparatus having shields angled inwardly toward a longitudinal centerline of said machine and supported on said frame rearwardly of said conveyor such that compost material discharged off said rearward end of said conveyor is engaged with said shields to consolidate said discharged compost material into an elongated pile centrally behind said machine.

11. In a machine for mixing and aerating an elongated windrow of compost material having a device for collecting shredded compost material from said windrow and conveying the shredded material rearwardly of said machine to form a pile of compost material rearwardly of said machine, the improvement comprising:
an air input apparatus directed a flow of air into said collected shredded compost material while being conveyed rearwardly of said machine to enhance aeration of the shredded compost material before being placed into said pile rearwardly of said machine.

12. The machine of claim 11 wherein said air input apparatus includes a pair of ducts directing said flow of air from outside said machine on opposing lateral sides thereof toward a longitudinal centerline of said machine, each said duct including a fan creating said flow of air.

13. The machine of claim 11 further comprising:
a mobile frame adapted for movement over the ground;
a central conveyor supported on said frame and oriented to convey shredded compost material rearwardly and upwardly for discharge therefrom into said pile rearwardly of said machine;
a header mounted forwardly on said frame to shred and collect said compost material from said elongated windrow, said header delivering collected compost material to said central conveyor, said air input apparatus directing said flow of air onto said central conveyor rearwardly of said header.

14. The machine of claim 13 wherein said header includes a transversely extending rotor having teeth mounted thereon to shred compost material from said windrow, said rotor being operable to convey said collected compost material to said conveyor.

15. The machine of claim 14 wherein said conveyor has a forward end and a rearward end with said forward end being mounted on said header, said header including a lift mechanism operable to move said header vertically, said front end of said conveyor moving vertically with said header, said rearward end being supported by a pair of laterally spaced support links, each said support link being pivotally connected to said conveyor and pivotally supported on said frame, said lift mechanism including a four-bar linkage and a hydraulic cylinder that induce a generally linear vertical movement of said header, said support links being operable to allow said rearward end moving longitudinally through the pivotal support provided by said support links.

16. The machine of claim 15 wherein each of said teeth is individually mounted on a rotor cylinder in an angled orientation with respect to a longitudinal centerline of said machine, said teeth being mounted on said rotor cylinder in a spiral configuration to facilitate movement of compost material through a central discharge opening in said header to said conveyor.

17. A compost mixing and aerating machine for engaging an elongated windrow of compost material, mixing and aerating said compost material, and re-depositing said compost material into a pile remote from said elongated windrow, comprising:
a mobile frame adapted for movement over the ground;
a central conveyor supported on said frame and oriented to convey material rearwardly and upwardly for discharge therefrom rearwardly of said conveyor, said conveyor having shielding defining an enclosed cavity around said central conveyor;
a header mounted forwardly on said frame, said header including a transverse rotor to shred and collect said compost material from said elongated windrow, said header further including a central discharge opening in register with said central conveyor, said rotor discharging said collected compost material through said central opening to said central conveyor; and
a pair of laterally disposed air ducts oriented angularly to a longitudinal centerline of said machine to direct a flow of air into said enclosed cavity of said central conveyor as compost material is being conveyed rearwardly thereby, each said air duct being associated with a fan to create said flow of air.

18. The machine of claim 17 wherein each said fan is mounted in the corresponding said duct at a corresponding inlet opening thereof to draw air from outside said machine into said enclosed conveyor cavity.

19. The machine of claim 17 wherein said rotor includes a plurality of teeth mounted thereon to shred said compost material from said windrow, said rotor being operable to convey said compost material to said conveyor, each of said teeth being individually mounted on a rotor cylinder in an angled orientation with respect to a longitudinal centerline of said machine to deflect compost material toward said central opening, said teeth being mounted on said rotor cylinder in a spiral configuration to facilitate movement of compost material through said central discharge opening to said conveyor.

20. The machine of claim 17 wherein said central conveyor has a forward end and a rearward end with said forward end being mounted on said header, said header including a lift mechanism operable to move said header vertically, said front end of said conveyor moving vertically with said header, said rearward end being mounted on a pair of laterally spaced support links, each said support link being pivotally connected to said conveyor and pivotally supported on said frame, said lift mechanism including a four-bar linkage and a hydraulic cylinder that induce a generally linear vertical movement of said header, said support links being operable to allow said rearward end moving longitudinally through the pivotal support provided by said support links.
